(19) United States
(12) Patent Application Publication
(10) Pub. No.: US 2023/0227505 A1
(43) Pub. Date: Jul. 20, 2023

(54) IDENTIFICATION AND APPLICATION OF ALV-J MHC-B2 RESTRICTIVE EPITOPE PEPTIDE

(71) Applicant: South China Agricultural University, Guangzhou (CN)

(72) Inventors: MANMAN DAI, GUANGZHOU (CN); QINGXIN WU, GUANGZHOU (CN); ZIWEI LI, GUANGZHOU (CN); MING LIAO, GUANGZHOU (CN); ZHIHAO AN, GUANGZHOU (CN); YUQIN ZHENG, GUANGZHOU (CN)

(21) Appl. No.: 17/831,388

(22) Filed: Jun. 2, 2022

(30) Foreign Application Priority Data
Jan. 14, 2022 (CN) 202210041210X

(51) Int. Cl. C07K 14/005; G01N 33/569

(52) U.S. Cl. CPC C07K 14/005; G01N 33/56983; C12N 2740/11034; C12N 2740/11022; G01N 2333/15

(57) ABSTRACT

An identification and an application of an ALV-J MHC-B2 restrictive epitope peptide are provided, which belong to the field of genetic engineering. An amino acid sequence of the provided ALV-J MHC-B2 restrictive epitope peptide is selected from SEQ ID NO:1, SEQ ID NO:2 and SEQ ID NO:3. An application of the ALV-J MHC-B2 restrictive epitope peptide in preparing an ALV epitope-based vaccine is provided. Restrictive motif of B2-haplotype chicken MHC class I molecule binding peptides is identified by in vitro elution assay. Potential epitopes in four proteins expressed by ALV-J are systematically screened by motif. The immunogenic B2-haplotype chicken ALV-J T-cell epitope peptides are identified by functional validation. It provides a material and theoretical basis for the research and development of ALV epitope-based vaccines.

### CROSS-REFERENCE TO RELATED APPLICATION

This application claims priority to Chinese Patent Application No. 202210041210X, filed on Jan. 14, 2022, the contents of which are hereby incorporated by reference.

### STATEMENT REGARDING SEQUENCE LISTING

The sequence listing associated with this application is provided in text format in lieu of a paper copy and is hereby incorporated by reference into the specification. The name of the text file containing the sequence listing is 77895_updated_SL. The text file is 14806 bytes; was created on Jun. 28, 2022; contains no new matter; and is being submitted electronically via EFS-Web.

### TECHNICAL FIELD

The disclosure relates to the technical field of genetic engineering, and more particularly to an identification and an application of a J-subtype avian leukosis virus (ALV-J) B2-haplotype major histocompatibility complex (MHC-B2) restrictive epitope peptide.

### BACKGROUND

Avian leukosis virus (ALV) is an avian retrovirus belonging to the alpharetrovirus genus, family retroviridae. It can be classified into 11 subtypes according to hosts and antigenic differences of viral envelope proteins. Among the subtypes, J-subtype ALV (ALV-J), which causes myeloid leukaemia and haemangiomas among chickens and has caused huge economic losses to the poultry industry since being discovered, contains coding regions and non-coding regions on its genome, same as other subtypes. There are mainly three genes in the coding region, namely capsid protein gene (gag), polymerase gene (pol) and envelope glycoprotein gene (env), and four proteins including Gag, Pol, Gp85 and Gp37 are expressed.

Major histocompatibility complex class I (MHC-I) expresses on the surface of all nucleated cells, and primarily works to present intracellular digestion-edited antigenic peptides for recognition performed by downstream cluster of differentiation 8-positive (CD8+) T cells, so as to activate body's cellular immunity to clear abnormal cells. The presented antigenic peptide is called cytotoxic T cell (CTL) epitope, which can be used as a basis to developed vaccines, and the developed vaccines can generate high levels of cellular immunity in the body, making it important for developing vaccines for various livestock and poultry diseases. Besides, it is reported that CD8+ T cells play a key role in resisting ALV infection, indicating great significance of developing epitope-based vaccines that can stimulate body's cellular immunity in preventing and controlling ALV. However, only a few reports on ALV epitope-based vaccines are available.

Epitope-based vaccines require immunogenic peptide epitopes, where the binding of MHC class I molecules to peptides is a prerequisite for the immunogenicity of peptides, indicating a significance of studying restrictive motifs of MHC class I molecule binding epitopes for developing epitope-based vaccines. MHC class I molecules are polymorphic, and have different restrictive motifs against different haplotypes. At present, only the motifs of B4, B12, B15, B19 and B21 haplotypes of chicken MHC class I molecules have been reported, and there are no reports on motif of B2-haplotype. However, compared with other haplotypes, MHC class I molecular B2-haplotype has a better ability to resist ALV, making it a good material for ALV experimental research and vaccine development. Therefore, it is necessary to screen and identify the restrictive motifs of B2-haplotype chicken MHC class I molecule binding peptides to systematically screen and obtain the immunogenic peptide epitopes expressed by ALV-J.

### SUMMARY

In view of the problems in the prior art described above, an identification and an application of a J-subtype avian leukosis virus (ALV-J) B2-haplotype major histocompatibility complex (MHC-B2) restrictive epitope peptide are therefore provided. The identification method of the disclosure eventually obtains ALV-J T-cell epitope peptides with significant immunogenicity in B2-haplotype chicken; providing material and theoretical basis for developing ALV epitope-based vaccines, the disclosure is of great significance for preventing and controlling ALV.

In order to achieve the objectives, the disclosure provides technical solutions as follows.

Specifically, the disclosure provides an ALV-J MHC-B2 restrictive epitope peptide, and the ALV-J MHC-B2 restrictive epitope peptide has an amino acid sequence selected from a group consisting of TVDTASSAI (SEQ ID NO:1), FVDFANRLI (SEQ ID NO:2) and SALQAFREV (SEQ ID NO:3).

The disclosure also provides an application of the ALV-J MHC-B2 restrictive epitope peptide in preparing an ALV epitope-based vaccine.

Moreover, the disclosure provides a method for identifying the ALV-J MHC-B2 restrictive epitope peptide, including the steps as follows:

(1) obtaining an MHC class I molecular heavy chain protein and an MHC class I molecular light chain protein by using a prokaryotic expression system, mixing the MHC class I molecular heavy chain protein, the MHC class I molecular light chain protein and a random peptide library and reacting to obtain an MHC class I-peptide complex;

(2) eluting the MHC class I-peptide complex obtained in the step (1) to obtain a peptide, sequencing and analyzing the peptide by a mass spectrum to determine a peptide restrictive epitope motif, and screening an ALV-J virus protein sequence according to the peptide restrictive epitope motif to obtain candidate peptides; and

(3) detecting immunogenicity of the candidate peptides in the step (2) by using a B2-haplotype animal model material infected with ALV-J virus, where the candidate peptides with the immunogenicity are obtained as the ALV-J MHC-B2 restrictive epitope peptides.

In an embodiment, a molar ratio of the MHC class I molecular heavy chain protein:the MHC class I molecular light chain protein:the random peptide library in step (1) is in a range of 1:1:(3-7).

In an embodiment, that random peptide library in the step (1) includes one selected from a group consisting of an octapeptide library, a nonapeptide library and a decapeptide library.

In an embodiment, the mixing the MHC class I molecular heavy chain protein, the MHC class I light chain protein, and a random peptide library and reacting in the step (1) is specifically as follows: adding the random peptide library into the MHC class I molecular light chain protein for reaction for 30 minutes (min) after renaturation of the WIC class I molecular light chain protein, and then adding the WIC class I molecular heavy chain protein for renaturation.

In an embodiment, a second position of an N-terminal of the peptide restrictive epitope motif in the step (2) is one of alanine (A or Ala) and valine (V or Val), and a ninth position of the N-terminal of the peptide restrictive epitope motif in the step (2) is one of valine (V or Val), isoleucine (I or Ile) and Leucine (L or Leu).

In an embodiment, the detecting immunogenicity of the candidate peptides in the step (2) by using a B2-haplotype animal model material infected with ALV-J virus in step (3) includes: performing an enzyme-linked immune absorbent spot (ELISpot) assay to obtain the candidate peptides with immunogenicity by using the B2-haplotype animal model material infected with the ALV-J virus.

The disclosure discloses that following technical effect:

the disclosure aims to determine a restrictive motif of B2-haplotype chicken WIC class I molecule binding peptide through an in vitro binding elution experiment, and therefore determine the motif screening candidate epitope peptides as X-A/V-X-X-X-X-X-X-V/I/L; potential epitopes in four proteins expressed by ALV-J are then screened by using a motif systematically, then the immunogenicity of the candidate peptides are detected through an in vitro ELISpot assay, and finally a peptide epitope with significant immunogenicity is determined. The amino acid sequence of the peptide epitope is TVDTASSAI, FVDFANRLI or SALQAFREV. The disclosure identifies ALV-J T cell epitope peptide of chicken B2-haplotype with immunogenicity for the first time, provides material and theoretical basis for researching and developing ALV epitope-based vaccine and has important significance for preventing and controlling ALV.

### BRIEF DESCRIPTION OF DRAWING

In order to explain embodiments of the disclosure or the technical solutions more clearly in the prior art, the drawings needed to be used in the embodiments are briefly introduced below, and it is obvious that the drawings in the following description are only some embodiments of the disclosure, and other drawings can be obtained according to the drawings by those skilled in the art without paying creative labor.

FIG. 1 illustrates changes in viremia of chickens after challenge.

FIG. 2 illustrates virus shedding of cloaca in chicken after challenge.

FIG. 3 illustrates changes in the proportion of cluster of differentiation 8-alpha-positive (CD8α+) T cells after challenge.

FIG. 4 illustrates changes in the proportion of CD4+ T cells after challenge.

FIG. 5 illustrates changes in cellular immunity-related genes in PBMC (peripheral blood mononuclear cell) after challenge.

FIG. 6A illustrates expression of chicken BF2*0201 (B2-haplotype chicken major histocompatibility complex heavy chain), where M represents a marker, 1 represents an uninduced holobacteria, 2 represents an induced holobacteria, 3 represents a post-induction supernatant, 4 represents a post-quassation supernatant, and 5 represents a fragmented precipitate.

FIG. 6B illustrates expression of chicken β2m (B2-haplotype chicken major histocompatibility complex light chain), where M represents a marker, 1 represents an uninduced holobacteria, 2 represents an induced holobacteria, 3 represents a post-induction supernatant, 4 represents a post-quassation supernatant, and 5 represents a fragmented precipitate.

FIG. 6C illustrates purification of chicken BF2*0201 and β2m by SDS-PAGE (sodium dodecyl sulfate-polyacrylamide gel electrophoresis), where M represents a marker, 1 represents a purified BF2*0201, and 2 represents a purified β2m.

FIG. 7A illustrates an elution effluent diagram of molecular sieve chromatography after renaturation.

FIG. 7B illustrates an SDS-PAGE identification diagram of elution components, where M represents a marker; 1 represents an elution peak at approximately 11 mL of elution volume; 2 represents an elution peak at approximately 15 mL of elution volume; 3 represents a target elution peak (at approximately 15.5 mL elution volume); and 4 represents an elution peak at approximately 18 mL of elution volume.

FIG. 8A illustrates an elution outflow diagram of ion exchange chromatography after molecular sieve chromatography.

FIG. 8B illustrates an SDS-PAGE identification diagram of elution components, where M represents a marker; 1 represents a target elution peak (where a concentration of NaCl is about 18%).

FIG. 9 illustrates a total ion chromatogram of an eluted peptide.

FIG. 10 illustrates a Base peak diagram of the eluted peptide.

FIG. 11 illustrates a result of de novo analysis of the eluted peptide portion.

FIG. 12 illustrates a weblogo diagram showing a motif identification result of B2-haplotype chicken MEW class I.

FIG. 13 illustrates a spot image of an enzyme-linked immune absorbent spot (ELISpot) assay.

FIGS. 14A-14C illustrate results of three ELISpot independent repeat experiments for IFN-gamma (interferon-γ) detection.

### DETAILED DESCRIPTION OF EMBODIMENTS

Now various exemplary embodiments of the disclosure are described in detail, which should not be construed as limiting the application, but rather as a more detailed description of certain aspects, features, and embodiments of the disclosure.

It is to be understood that terms used herein are for the purpose of describing embodiments only and are not intended to be limiting of the disclosure. In addition, for numerical ranges in the disclosure, it is understood that each intermediate value between the upper and lower limits of the range is also specifically disclosed. Each smaller range between any stated value or stated range of intermediate values and any other stated value or intermediate value within the stated range is also included within the disclosure. The upper and lower limits of these smaller ranges may independently be included or excluded from the range.

Unless otherwise indicated, all technical and scientific terms used herein have the same meaning as commonly understood by those skilled in the art to which the disclosure relates. While the disclosure describes only the preferred methods and materials, any methods and materials similar or equivalent to those described herein may be used in the practice or testing of the disclosure. All documents mentioned in this specification are incorporated by reference to disclose and describe methods and/or materials related to the documents. In case of conflict with any incorporated literature, the content of this specification shall prevail.

It will be apparent to those skilled in the art that various modifications and changes can be made to the specific embodiments of the disclosure without departing from the scope or spirit of the disclosure. Other embodiments will be apparent to those skilled in the art from the description of the disclosure. The specification and embodiments of the disclosure are exemplary only.

As used herein, the terms “comprising,” “including,” “having,” “containing,” and the like are open ended terms that mean including, but not limited to.

### Explanation of Terms

ALV-J: J-subtype avian leukosis virus;

MHC-I: Major histocompatibility complex class I;

BF2*0201: B2-haplotype chicken major histocompatibility complex heavy chain, also referred to as MHC class I heavy chain;

β2m: B2-haplotype chicken major histocompatibility complex light chain, also referred to as MHC class I light chain;

ELISpot: enzyme-linked immune absorbent spot assay;

PBMC: peripheral blood mononuclear cell;

LC-MS/MS: high performance liquid chromatography with tandem mass spectrometry;

De novo sequencing: sequencing from scratch;

SPF chicken: specific-pathogen-free chicken;

CTL: cytotoxic T cell; and

IFN-γ: interferon-gamma.

1 Test Materials

1.1 Animals and Viruses for the Test

The chickens used in this experiment are 4-week-old BW/G3 (B2-haplotype) SPF chickens purchased from the National Laboratory Poultry Animal resource center of China. ALV-J SCAU-HN06 strain is stored in the National Local Joint Engineering Laboratory for Prevention and Control of Human and Veterinary Diseases, South China Agricultural University.

1.2 Main Reagents for the Test

Trihydroxymethyl aminomethane (Tris-base), L-arginine (L-Arg), reduced glutathione (GSH), oxidized glutathione (GSSG), isopropyl thiogalactoside (IPTG), guanidine hydrochloride and dithiothreitol (DTT) are purchased from Amresco, the USA. Sodium chloride, ethylenediaminetetraacetic acid disodium (EDTA-2Na), Coomassie brilliant blue R-250 and LB liquid culture medium dry powder are purchased from Solarbio, China. RPMI-1640 medium, FBS Australian fetal bovine serum, L-glutamine (100×), and β-mercaptoethanol are purchased from GIBCO, the USA. Dimethyl sulfoxide (DMSO), saponin, and Brefeldin A (BFA) are purchased from Sigma, the USA. Phorbol ester and ionomycin (PMA+Ionomycin), and special color developing solutions of TMB (tetramethylbenzidine) ELISpot are purchased from Dakewe, China. Chicken IFN-γ ELISpot^{BASIC} kit is purchased from Mabtech. Anti-chicken CD3 anti-body, anti-chicken CD4 anti-body, anti-chicken CD8α anti-body, and anti-chicken IFN-γ-FITC antibodies are purchased from SouthernBiotech. The chicken peripheral blood lymphocyte separation solution kit, chicken organ tissue mononuclear cell separation solution kit and red blood cell lysate are purchased from Tianjin Haoyang Biology Co., Ltd. ChamQ SYRB qPCR Master Mix is purchased from Vazyme, Nanjing, China.

1.3 Preparation of Main Solution

Washing Buffer: 0.5% Triton-100, 50 mM Tris pH 8.0, 300 mM NaCl, 10 mM EDTA, 1‰ β-mercaptoethanol.

Resuspension Buffer: 50 mM Tris pH 8.0, 100 mM NaCl, 10 mM EDTA, 1‰ β-mercaptoethanol.

Dissolution Buffer: 6 M Gua-HCl, 10% glycerol, 50 mM Tris pH 8.0, 100 mM NaCl, 10 mM EDTA, 10 mM DTT.

Refolding Buffer: 100 mM Tris pH 8.0, 400 mM L-Arg HCl, 2 mM EDTA, 5 mM GSH, 0.5 mM GSSG, 0.5 mM PMSF (phenylmethanesulfonyl fluoride).

Molecular sieve buffer: 20 mM Tris-HCl (pH 8.0), 50 mM NaCl.

Ion exchange solution A: 10 mM Tris-HCl (pH 8.0), 10 mM NaCl.

Ion exchange solution B: 10 mM Tris-HCl (pH 8.0), 1 M NaCl.

2 Test Methods

2.1 Establishment of B2-Haplotype Chicken Model Infected with ALV-J

Twelve 4-week-old B2-haplotype chickens are randomly separated into a challenge group and a control group, with six in each group. In the challenge group, 1 mL ALV-J is injected intraperitoneally, and the virus titer is 10^{4.7} TCID₅₀/100 μL. In the control group, 1 mL PBS (phosphate buffer solution) is injected intraperitoneally. After virus tapping, corresponding samples are collected for the following testing:

2.1.1 Detection of Cloacal Virus Shedding of B2-Haplotype Chicken after Infection

Cloacal swabs are collected and added to 1 mL of PBS for cryopreservation and restored to room temperature before testing. Virus shedding is detected by ELISA (enzyme-linked immunosorbent assay) using the avian leukosis antigen detection kit from IDEXX. The coating plate is provided with positive and negative control wells and sample wells. According to the kit requirements, 100 μL of corresponding standard substance is added to the positive and negative control wells, and 100 μL of cloacal swab solution is added to the sample wells. After reacting at 18-26° C. for 60 min, 350 μL distilled water is added to each well for washing, and the washing is repeated five times, followed by 100 μL enzyme-labeled antibody added to each well. After reacting for 60 min at 18-26° C., the washing procedure is the same as above and repeated five times, and then 100 μL TMB substrate solution is added to each well. After the reaction at 18-26° C. for 15 min, 100 μL stop solution is added to each well to stop the reaction, and the absorbance value A of each sample well and the positive and negative control wells at 650 nm is measured and recorded.

2.1.2 Detection of Changes in B2-Haplotype Chicken Viremia after Infection

The DF-1 cells are plated in a 24-well plate, and when the cells grow to 90%, 500 μL of chicken peripheral blood is collected into anticoagulation and then subjected to centrifugation at 2,000 g for 8 min at 4° C. to separate plasma. 100 μL of plasma is inoculated with DF-1 cells, and the cells are cultured in an incubator at 37° C. for 24 hours, then the medium is replaced with 1640 medium containing 2% fetal bovine serum, and the culture is continued for five days. On the 6^{th}-7^{th} days, the cell culture supernatant is taken for ELISA assay to detect viral antigen, and the steps are the same as those in 2.1.1.

2.1.3 Detection of Changes in T Cell Subtypes in PBMC of Infected B2-Haplotype Chickens

After the peripheral blood lymphocytes are isolated by a conventional method and counted by trypan blue staining, 1*10⁶ cells from each sample are placed in flow cytometry tube and stained with anti-chicken CD3, CD4, and CD8α flow cytometry antibodies; a channel control group and a blank control group are set at the same time. Each tube is then added with PBS to 1 mL, and subjected to centrifugation at 400 g for 5 min. The supernatant is discarded. Each antibody is diluted in accordance with the instruction proportion with flow buffer, and 100 μL diluted antibody is added to each sample to resuspend the cells, and the cells are incubated at 4° C. for 30 min. After incubation, each tube is added with PBS to a final volume of 1 mL, and the samples are centrifuged again at 400 g for 5 min. The PBS is discarded, and each tube is added with 200 μL flow buffer to resuspend the cells, and the cells are tested.

2.1.4 Detection of Cellular Immunity-Related Genes Changes in B2-Haplotype Chicken PBMC after Infection

RNA extraction from peripheral blood lymphocytes isolated in step 2.1.3 by TRIZOL method includes steps as follows. 1 mL TRIZOL is added to the cells, mixed and shaken to split the cells, then 200 μL chloroform is added, and evenly mixed by shaking to obtain a mixture. After standing at room temperature for 10 min, the mixture is centrifuged at 12,000 g for 15 min at 4° C., then the transparent supernatant is sucked and transferred into a new enzyme-free centrifuge tube. An equal volume of isopropanol is added, and mixed evenly by turning it upside down for reaction at −20° C. After 30 min, the sample is taken out and centrifuged at 12,000 g for 10 min at 4° C., and RNA precipitation is observed. The supernatant is discarded, and 1 mL of 75% ethanol is added to the RNA precipitation. After shaking, the RNA precipitation mixed with ethanol is centrifuged at 12,000 g for 10 min at 4° C. to wash RNA. After two replicates, the supernatant is discarded, and the nozzle of the tube is opened to allow natural volatilization of ethanol. Finally, 30 μL of 0.1% DEPC (diethylpyrocarbonate) water preheated to 65° C. in advance is added to the precipitate to dissolve RNA. A spectrophotometer is used to measure the concentration of RNA.

RNA reverse transcription is performed using PrimeScript™ RT Master Mix (Perfect Real Time) kit from TAKARA company. The system is shown in Table 1, and the reaction procedure is: reaction at 37° C. for 15 min and inactivation at 85° C. for 5 seconds.

TABLE 1
Reverse Transcription System

| Reagent | Volume |
|---|---|
| 5 × PrimeScript RT | 4 μL |
| Master Mix | |
| Rnase Free dH₂O | 14 μL |
| RNA | 2 μL |

The reverse-transcribed cDNA is subjected to fluorescent quantitative PCR amplification to detect changes in cellular immunity-related genes in PBMC. The target genes and detecting primers are shown in Table 2, and the reaction system is shown in Table 3. Reaction procedure: pre-denaturation at 95° C., 30 s; cycle reaction at 95° C. for 10 s, 60° C. for 30 s for a total of 40 cycles. Melting curve analysis is carried out at 95° C., 15 s, 60° C., 60 s, 95° C., 15 s.

TABLE 2
qPCR Primers of Cell Immunity Related Genes

| Gene | Primer sequence (5′-3′) | |
|---|---|---|
| CAPDL | F-Primer | GAACATCATCCCAGCGTCCA |
| | | (SEQ ID NO: 77) |
| | R-Primer | CGGCAGGTCAGGTCAACAAC |
| | | (SEQ ID NO: 78) |
| Granzyme K | F-Primer | CGGGAAGCAACTGTTGAAAT |
| | | (SEQ ID NO: 79) |
| | R-Primer | GAGTCTCCCTTGCAAGCATC |
| | | (SEQ ID NO: 80) |
| Perforin | F-Primer | ATGGCGCAGGTGACAGTGA |
| | | (SEQ ID NO: 81) |
| | R-Primer | TGGCCTGCACCGGTAATTC |
| | | (SEQ ID NO: 82) |
| IFN-γ | F-Primer | CCTCCAACACCTCTTCAACATG |
| | | (SEQ ID NO: 83) |
| | R-Primer | TGGCGTGCGGTCAAT |
| | | (SEQ ID NO: 84) |
| TNF-α | F-Primer | GCTGTTCTATGACCGCCCAGTT |
| | | (SEQ ID NO: 85) |
| | R-Primer | AACAACCAGCTATGCACCCCA |
| | | (SEQ ID NO: 86) |
| IL-1β | F-Primer | GGTCAACATCGCCACCTACA |
| | | (SEQ ID NO: 87) |
| | R-Primer | CATACGAGATGGAAACCAGCAA |
| | | (SEQ ID NO: 88) |
| IL-2 | F-Primer | GCTAATGACTACAGCTTATGGAGCA |
| | | (SEQ ID NO: 89) |
| | R-Primer | TGGGTCTCAGTTGGTGTGTAGAG |
| | | (SEQ ID NO: 90) |
| NK lysin | F-Primer | GATGGTTCAGCTGCGTGGGATGC |
| | | (SEQ ID NO: 91) |
| | R-Primer | CTGCCGGAGCTTCTTCAACA |
| | | (SEQ ID NO: 92) |
| HMG-2 | F-Primer | AGAGCACAAGAAGAAGCAC |
| | | (SEQ ID NO: 93) |
| | R-Primer | GTCTTTTAGGAGCGTTGGGGTC |
| | | (SEQ ID NO: 94) |
| MHC-I | F-Primer | AAGAAGGGGAAGGGCTACAA |
| | | (SEQ ID NO: 95) |
| | R-Primer | AAGCAGTGCAGGCAAAGAAT |
| | | (SEQ ID NO: 96) |

TABLE 3
Fluorescence Quantitative System

| Reagent | Volume |
|---|---|
| 2 × ChamQ Universal | 10 μL |
| SYBR qPCR Mix | |
| F-Primer | 0.4 μL |
| R-Primer | 0.4 μL |
| cDNA | 2 μL |
| ddH₂O | 7.2 μL |

2.2 Identification of B2-Haplotype Chicken MHC Class I Molecular Binding Peptides Motif

2.2.1 Synthesis of Random Nonapeptide Library

Peptide library is synthesized by Beijing SciLight Biotechnology Co., Ltd. Using Fomc method, alkoxy benzyl alcohol type resins as carriers are crosslinked with 19 kinds of α-amino protecting group amino acids except cysteine firstly, then all the carriers are combined and subjected to amino deprotection, and triethylamine is used to neutralize the free amino acids. After sufficient washing, the mixture is divided into 19 aliquots. A new batch of amino acids is activated, and an excess of each of the 19 amino acids is added to the 19 aliquots for condensation. After a total of 8 replicates, 90% trifluoroacetic acid is used to excise the carrier and a reverse column is used to perform purification to obtain a random nonapeptide library.

Quality inspection: the distribution of each amino acid is analyzed for quality inspection by subjecting the synthetic random nonapeptide library to LC-MS/MS (high performance liquid chromatography tandem mass spectrometry) and de novo analysis. The LC-MS/MS analysis procedures are as follows: the obtained random nonapeptide library is redissolved with 20 μL of 0.1% formic acid/water solution, 10 μL of sample is injected, and the samples are separated on a chromatographic column (C18, 3 μm, 100 μm, 100 Å, and 75 μm*15 cm), where mobile phase A is 0.1% formic acid water solution, and mobile phase B is 0.1% formic acid acetonitrile solution. The chromatographic gradients are shown in Table 4, and the mass spectrometer parameters are arranged as shown in Table 5. De novo analysis is performed on the collected data, and the software parameter settings are shown in Table 6.

TABLE 4
Chromatographic Gradients

| Duration | Mobile phase | Mobile phase | Flow rate |
|---|---|---|---|
| (minute) | A (%) | B (%) | (nL/min) |
| 0 | 99 | 1 | 400 |
| 1 | 95 | 5 | 400 |
| 90 | 70 | 30 | 400 |
| 92 | 60 | 40 | 400 |
| 95 | 10 | 90 | 400 |
| 105 | 10 | 90 | 400 |
| 106 | 99 | 1 | 400 |
| 125 | 99 | 1 | 400 |

TABLE 5
Mass Spectrometer Parameter Settings

| Parameters | Parameter setting |
|---|---|
| Mass spectrometer | Thermo Scientific |
| | Q Exactive HF |
| Spray voltage | 2.0 kV |
| Capillary temperature | 320° C. |
| S-lens RF Level | 55 |
| Collision energy | NCE 27 |
| Isolation Window | 2.2 m/z |
| Primary resolution | 70,000 @ m/z 200 |
| Secondary resolution | 17,500 @ m/z 200 |
| Parent ion scanning range | m/z 200-1800 |
| Sub-ion scanning range | Automatic |
| MS1 AGC and ion | 3e6, 60 ms |
| implantation time | |
| MS2 AGC and ion | 5e4, 50 ms |
| implantation time | |
| Data-dependent MS/MS | Top 10 |
| Dynamic exclusion time | 10 s |

TABLE 6
De Novo Software Analysis Parameter Settings

| Parameters | Parameter setting |
|---|---|
| Enzyme | No-Specific |
| Variable modifications | Oxidation (M), |
| | Deamidated (N, Q) |
| Peptide Mass Tolerance | ±10 ppm |
| Fragment Mass Tolerance | 0.02 Da |

2.2.2 Acquisition of MHC Class I Molecular Heavy and Light Chain Expression Strains

The prokaryotic expression vectors of the two genes are mainly constructed by Wuhan Genecreate Co., Ltd. The CDS sequences of heavy chain BF2*0201 (GenBank: AY234770.1) and light chain (32m (GenBank: AB178590.1) of B2-haplotype chicken MHC class I molecule are downloaded from NCBI, and the transmembrane region and signal peptide of the sequences are predicted by TMHMM and SignalP tools, respectively. The CDS sequences of BF2*0201 and light chain β2m (GenBank: AB178590.1) after removal of the signal peptide and transmembrane region are obtained respectively, and the transmembrane region and signal peptide are predicted by TMHMM and SignalP tools again respectively to confirm the accuracy. The DNA sequences of BF2*0201 and β2m after removal of the signal peptide and transmembrane region are respectively synthesized, and then the two DNA sequences are inserted into the pET21a (+) vector through two enzyme cleavage sites of Nde I and Hind III, respectively; sequencing with no error is considered a successful construction.

The constructed vectors are respectively converted into BL21 competent cells according to the conventional method of molecular cloning, and then single colonies are picked and sent for sequencing.

2.2.3 Expression and Purification of Heavy and Light Chain Proteins of MHC Class I Molecule

2.2.3.1 Identification of Heavy and Light Chain Proteins Expression Forms of MHC Class I Molecule

The bacteria solutions sequenced correctly are inoculated into 5 mL of resistant LB at a ratio of 1:100 respectively for small amounts of test expression of the protein. When the medium is cultured under shaking at 37° C. with a shaking rate of 200 r/min until the OD₆₀₀ value in the medium is within the range of 0.4-0.6, the culture is stopped. Uninduced bacterial solution of 2 mL is sucked and taken as the negative control, and the rest of the bacterial solutions are added with 1 mM IPTG for induction as the experimental group. After the bacterial solutions of the control group and the experimental group are cultured at 37° C. for 5 h at a shaking speed of 200 r/min, 1 mL of the bacterial solution is sucked from the experimental group to be labeled as induced holobacteria, the remaining bacterial solution is centrifuged at the highest rotation speed of a centrifuge for 2 min, a part of the supernatant is sucked to be labeled as the induced supernatant, and the remaining supernatant is then discarded; the precipitate is re-suspended in a 1.5 mL EP tube with 700 μL PBS, and the suspension is ultrasonically crushed for 20 cycles according to a program of 4 s of work, 4 s of pause and 3 W of power. Then, the samples are centrifuged at the highest rotation speed for 10 min, and part of the supernatant is labeled as the post-quassation supernatant. The remaining supernatant is discarded, and the precipitate is resuspended with 700 μL PBS, and labeled as the fragmented precipitate. Then, 20 μL of all labeled samples are mixed with 5 μL of 5×SDS loading buffer in a boiling water bath for 10 min for further analysis.

The commercial polyacrylamide gel is used for electrophoresis identification. After the equipment is assembled, 10 μL of prepared sample is orderly added to each well, and protein Marker is added at the same time. The program is started at 80 V firstly, and when the sample migrates to the separation gel, the voltage is increased to 120 V and the procedure is run for about 1 h (hour). After electrophoresis, the gel is stained with Coomassie brilliant blue staining solution overnight at room temperature. On the next day, the decolorizing solution is used for decolorization until a clear protein band appeared on the gel. The protein expression form is determined by the position of the band.

2.2.3.2 Expression and Purification of Heavy and Light Chain Inclusion Bodies of MHC Class I Molecule

The bacterial solution is inoculated into 2 L resistant LB at a ratio of 1:100, and oscillated for culture at 37° C. until OD₆₀₀=0.4-0.6. After that, IPTG is added to make the final concentration reach 1 mM, and the oscillation culture is continued for 5 h at 37° C. The following steps are all carried out on ice or at 4° C.: after the culture is completed, the thalli are collected and crushed by a cell high-pressure crusher. The supernatant is discarded after centrifugation at 10,000 g for 10 min, and the precipitate is oscillated and suspended in a pre-cooled Washing Buffer. Then, precipitate is subjected to centrifugation at 10,000 g for 10 min for washing, and the procedure is repeated for three times. Afterwards, pre-cooled re-suspension buffer is added for oscillation and re-suspension, and 10 μL sample preparation is taken for SDS-PAGE analysis. The rest samples are subjected to high-speed centrifugation at 10,000 g for 10 min, then the supernatant is discarded and the precipitate is weighed. The final concentration is set at 30 mg/mL by adding dissolution buffer, and it is magnetically stirred at 4° C. for complete dissolution.

2.2.4 In Vitro Renaturation and Purification of MHC Class I-Peptide Complex

The following steps are carried out at 4° C.: preparing and placing 1 L refolding buffer on a magnetic stirrer for gentle stirring; adding β2m inclusion body fluid of 1 mL dropwise to the refolding buffer, and after renaturation for 8 h, adding about 10 mg of random nonapeptide library dissolved in DMSO; after the reaction of 30 min, adding 3 mL BF2*0201 inclusion body fluid dropwise to the refolding buffer for renaturation overnight.

After renaturation, the sample is concentrated at 30 mL in an ultrafiltration concentration cup, centrifuged at 8,000 g for 10 min, and the supernatant is passed through a molecular sieve to replace the solvent of the sample with a molecular sieve buffer. Then the sample is highly concentrated to 1.5 mL in the ultrafiltration concentration tube. The sample is first purified by molecular sieve chromatography, and the target protein is collected and further purified by ion exchange chromatography column. At the same time, SDS-PAGE identification is performed after sampling.

2.2.5 Complex Peptide Elution

The purified complex is highly concentrated in an ultrafiltration concentration tube to a volume of less than 200 μL, and the sample is incubated with 400 μL 0.2 M acetic acid at 65° C. for 1 hour, then transferred to a 3 KD ultrafiltration concentration tube for centrifugation, and the lower filtrate is collected as the eluted peptide.

2.2.6 LC-MS/MS Mass Spectrometry Sequencing and De Novo Analysis

The procedures are the same as the quality inspection procedures in 2.2.1.

2.2.7 Data Analysis and Determination of Motifs

The peptides with the score of more than 50 points after de novo analysis are selected for statistics, and the coefficient of variation (Vs=σ/X) of each locus is calculated. Two sites with the largest values are the restriction sites. Meanwhile, a logo histogram is drawn by weblogo website (https://weblogo.berkeley.edu/logo.cgi) to visually display the distribution proportion of amino acids in each locus. The two amino acids with the highest proportion in the restriction sites are selected as the motif for screening the peptide.

2.2.8 Screening and Synthesis of Epitopes Based on Obtained Motif

The protein sequences of ALV-J HN06 strain are screened based on the motif determined in 2.2.7. As shown in Table 7, peptide is synthesized by Genscript Biotechnology Co., Ltd. by chemical method and purified by HPLC to achieve a purity of over 95%.

TABLE 7
ALV-J Epitopes Screened Based on Motif

| | | | | Subordinate |
|---|---|---|---|---|
| Protein sequence | | | | protein |
| PALTDWARI | EALMSFPLL | PVKQRSVYI | LASTGPPVV | Gag |
| (SEQ ID NO: 4) | (SEQ ID NO: 10) | (SEQ ID NO: 15) | (SEQ ID NO: 20) | |
| TAPLTDQGI | AALLRPGEL | VVDTANPQI | SALQAFREV | |
| (SEQ ID NO: 5) | (SEQ ID NO: 11) | (SEQ ID NO: 16) | (SEQ ID NO: 3) | |
| IAAAMSSAI | QAFREVARL | AVAMVRGSI | FANRLIKAV | |
| (SEQ ID NO: 6) | (SEQ ID NO: 12) | (SEQ ID NO: 17) | (SEQ ID NO: 21) | |
| TANPQIHGI | VAMVRGSIL | AALSQRAMV | DVTNLMRVI | |
| (SEQ ID NO: 7) | (SEQ ID NO: 13) | (SEQ ID NO: 18) | (SEQ ID NO: 22) | |
| PVVAMPVVI | MVLGKSGEL | LVAIAASAL | FVDFANRLI | |
| (SEQ ID NO: 8) | (SEQ ID NO: 14) | (SEQ ID NO: 19) | (SEQ ID NO: 2) | |
| GVINRDGSL | | | | |
| (SEQ ID NO: 9) | | | | |
| VARCEQGAI | SAPALEAGV | SAMAAVLHV | PVWIDQWPL | POL |
| (SEQ ID NO: 23) | (SEQ ID NO: 32) | (SEQ ID NO: 42) | (SEQ ID NO: 52) | |
| RALSKACNI | MAPRSWLAV | SALPRGWPL | AVQQGAPVL | |
| (SEQ ID NO: 24) | (SEQ ID NO: 33) | (SEQ ID NO: 43) | (SEQ ID NO: 53) | |
| EASPLFAGI | AVRTFGKEV | LAASSHDGL | VVGQVLEPL | |
| (SEQ ID NO: 25) | (SEQ ID NO: 34) | (SEQ ID NO: 44) | (SEQ ID NO: 54) | |
| TVDTASSAI | KVRVTDHPV | VAEPRIATL | QATFQAYPL | |
| (SEQ ID NO: 1) | (SEQ ID NO: 35) | (SEQ ID NO: 45) | (SEQ ID NO: 55) | |
| AVLGRPKAI | NVVTDSAFV | PALGIPPRL | EAKDLHTAL | |
| (SEQ ID NO: 26) | (SEQ ID NO: 36) | (SEQ ID NO: 46) | (SEQ ID NO: 56) | |
| GAVQQGAPV | IVVTQHGRV | MAWREIVQL | EAGVNPRGL | |
| (SEQ ID NO: 27) | (SEQ ID NO: 37) | (SEQ ID NO: 47) | (SEQ ID NO: 57) | |
| PARRFQWKV | TVLTEGPPV | TAWLEVLTL | MVERANRLL | |
| (SEQ ID NO: 28) | (SEQ ID NO: 38) | (SEQ ID NO: 48) | (SEQ ID NO: 58) | |
| PALPLEGAV | AALHLAIPL | IARPLHVSL | GVQYLGYKL | |
| (SEQ ID NO: 29) | (SEQ ID NO: 39) | (SEQ ID NO: 49) | (SEQ ID NO: 59) | |
| KAFTAWLEV | KASGSYRLL | EARAVAMAL | DVQKLVGSL | |
| (SEQ ID NO: 30) | (SEQ ID NO: 40) | (SEQ ID NO: 50) | (SEQ ID NO: 60) | |
| SAFVAKMLL | AAFILEDAL | RAVAMALLL | | |
| (SEQ ID NO: 31) | (SEQ ID NO: 41) | (SEQ ID NO: 51) | | |
| LASQTACLI | KALPPGIFL | YVNRSWTMV | TACQNDTDL | GP85 |
| (SEQ ID NO: 61) | (SEQ ID NO: 63) | (SEQ ID NO: 65) | (SEQ ID NO: 66) | |
| TAKALPPGI | NALGGPCYL | | | |
| (SEQ ID NO: 62) | (SEQ ID NO: 64) | | | |
| AAAQALREI | LVIVCLLAI | QALREIERL | LAKGVKTLL | GP37 |
| (SEQ ID NO: 67) | (SEQ ID NO: 70) | (SEQ ID NO: 73) | (SEQ ID NO: 75) | |
| AVLQNRAAI | FASFFAPGV | QANLTSLIL | GVKTLLFAL | |
| (SEQ ID NO: 68) | (SEQ ID NO: 71) | (SEQ ID NO: 74) | (SEQ ID NO: 76) | |
| RAAIDFLLL | FALLVIVCL | | | |
| (SEQ ID NO: 69) | (SEQ ID NO: 72) | | | |

2.3 Detection of Immunogenicity of Candidate Peptides by ELISpot Assay

The B2-haplotype chicken infected for 28 d (day) in 2.1 is taken, and the spleen lymphocytes of the chicken are isolated according to the kit instructions of the chicken organ tissue mononuclear cell isolation solution, and an ELISpot assay is performed to detect the immunogenicity of candidate peptide epitopes. The specific steps are as follows:

(1) PVDF 96-well plate activation: 15 μL 35% ethanol is added to each well for reaction for less than 1 min, and then the wells are washed five times with sterile water.

(2) Antibody coating: the anti-chicken IFN-γ is diluted to 15 μg/mL, and 100 μL of that is added to each well, and the mixture is incubated at 4° C. overnight.

(3) Blocking: the coating solution is discarded, followed by washing 5 times with DPBS, and the plate is dried on the sterilized absorbent paper. RMPI 1640 medium containing 10% FBS is added to PVDF 96-well plate at 200 μL/well and the reaction is blocked at room temperature for 30 min.

(4) Stimulation: the blocking solution is discarded and 100 μL of prepared lymphocyte suspension at the concentration of 5*10⁶ cells/mL is added to each well. At the same time, 50 μL diluted antigenic peptide is added to the experimental group at the final concentration of 10 μg/mL, PMA at the final concentration of 10 μg/mL is added to the positive control group, and no irritant is added to the negative control group. After all the samples are added, PVDF 96-well plate is placed in CO₂ incubator at 37° C. for 24-48 h.

(5) Incubation of secondary antibody: after the culture, the cells are shaken off and the plate is washed five times with DPBS. Then 100 μL biotinylated secondary antibody with a concentration of 1 μg/mL is added to each well and the plate is incubated for 2 h at room temperature.

(6) HRP incubation: the secondary antibody solution is discarded and washed with DPBS for 5 times. Then 100 μL streptavidin-labeled HRP is added to the well and incubated at room temperature for 1 h.

(7) Color development: the HRP incubation solution is discarded and the plate is washed with DPBS for 5 times. Then 100 μL TMB color-developing substrate is added to each well followed by incubation at room temperature or 37° C. for 15-30 min. When obvious spots appeared in the positive control group, the color-developing solution could be discarded. the color development is stopped with pure water and the plate is read after air drying.

3 Results

3.1 B2-Haplotype Chicken Viremia and Cloacal Virus Shedding after ALV-J Infection

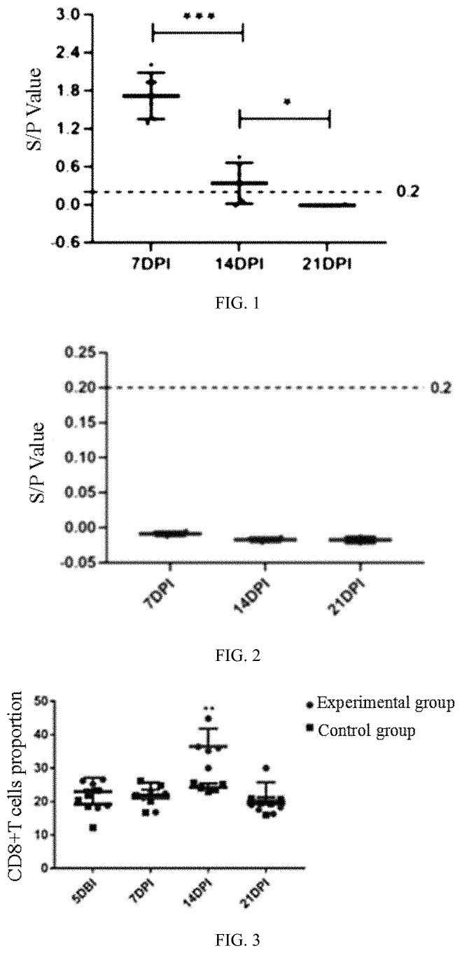

The changes in viremia of chickens after challenge are shown in FIG. 1. The virus content in blood is the highest at 7 DPI (the 7^{th} day post infection), indicating that all the chickens in the challenge group are successfully infected with ALV-J. The virus content in blood is significantly decreased (P<0.001) at 14 DPI, and no virus is detected at 21 DPI. The virus shedding of chicken cloaca after challenge is shown in FIG. 2, and no virus is observed.

3.2 Changes of T Cell Subtypes in B2-Haplotype Chicken PBMC after ALV-J Infection

The PBMC are collected from the peripheral blood of chickens on 7^{th}, 14^{th} and 21^{st} day after infection, and the proportion of CD8α⁺ T cells and CD4⁺ T cells is measured by flow cytometry. The changes of the proportion of CD8α⁺ T cells are shown in FIG. 3. There is no significant difference between the challenge group and the control group on 7 DPI, but a significant increase in the proportion of CD8α⁺ T cells in the challenge group is detected on 14 DPI, indicating that virus infection could induce CD8α⁺ T cell immunity in chickens. On 21 DPI, CD8α⁺ T cells in the challenge group are recovered to a normal level. The proportion of CD4+ T cells is shown in FIG. 4. Throughout the testing, there is no statistical difference in the proportion of CD4⁺ T cells in the challenge group compared to the control group.

3.3 Changes in Cellular Immunity-Related Genes Expressions of B2-Haplotype Chicken PBMC after Infection

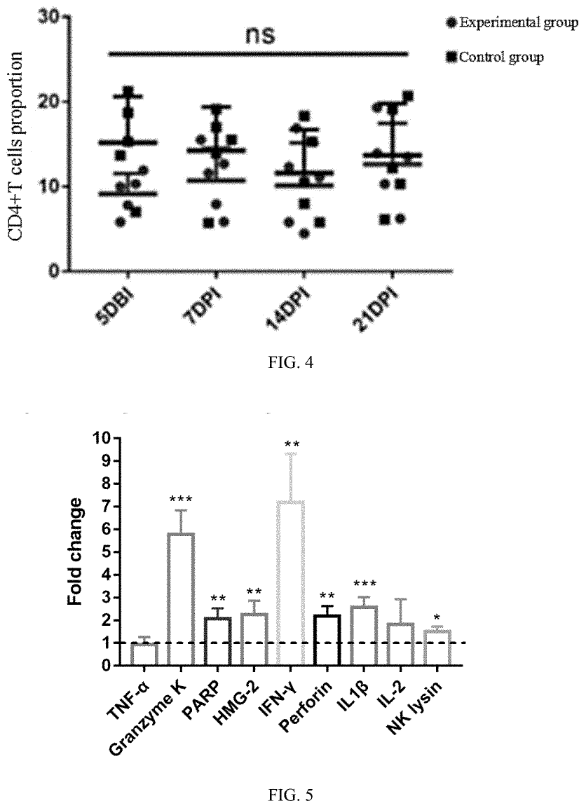

According to the results of FIG. 3, 14 DPI PBMC samples are selected to detect the expression of cellular immunity-related genes, and the changes in the related genes are shown in FIG. 5. In addition to TNF-α and IL-2, the selected cellular immunity-related genes for detection are significantly up-regulated compared with those in the control group, including NK lysin, Poly (ADP-ribose) polymerase 1 (PARP), High-mobility group-2 protein (HMG-2), Perforin, IFN-γ, IL-1β and Granzyme K. Combined with the increased proportion of CD8α⁺ T cells in 3.2, this further indicates that ALV-J infection successfully activated the cellular immunity of B2-haplotype chicken. In summary, the model of ALV-J SCAU-HN06 strain infecting BW/G3(B2-haplotype) SPF chicken and effectively inducing cellular immunity is successfully established.

3.4 Expression and Purification of Chicken BF2*0201 and β2m

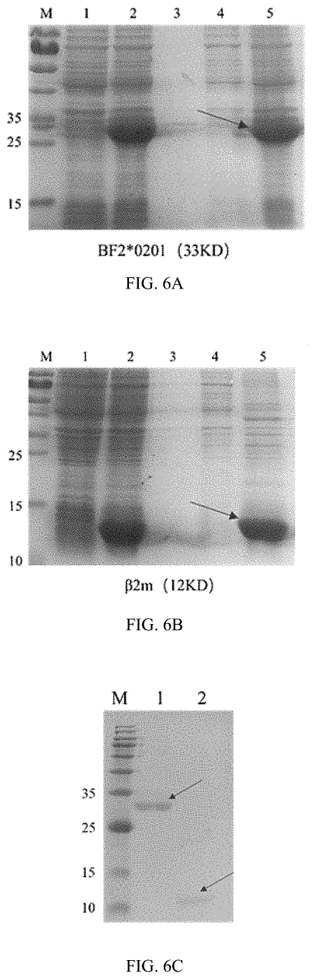

FIGS. 6A and 6B show that the expressions of proteins BF2*0201 and β2m are successful, and both proteins are mainly expressed as inclusion bodies. As shown in FIG. 6C, after a series of operations, the purification of the two proteins is effective.

3.5 In Vitro Renaturation and Purification of MHC Class I-Peptide Complex

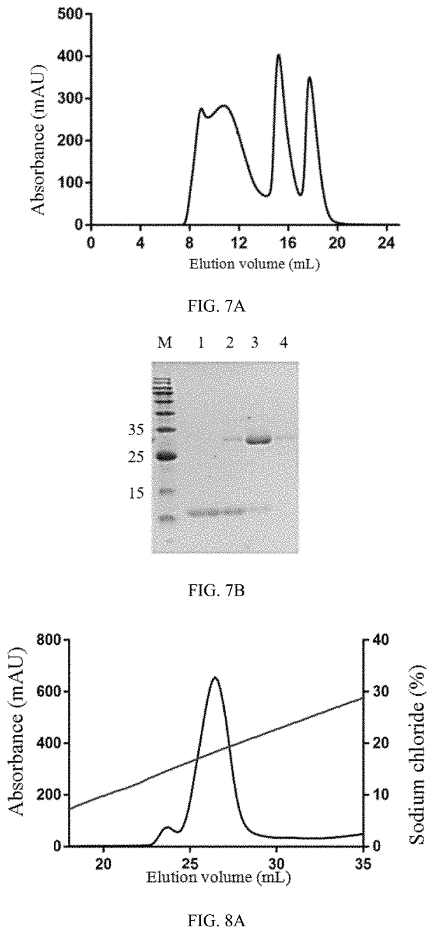

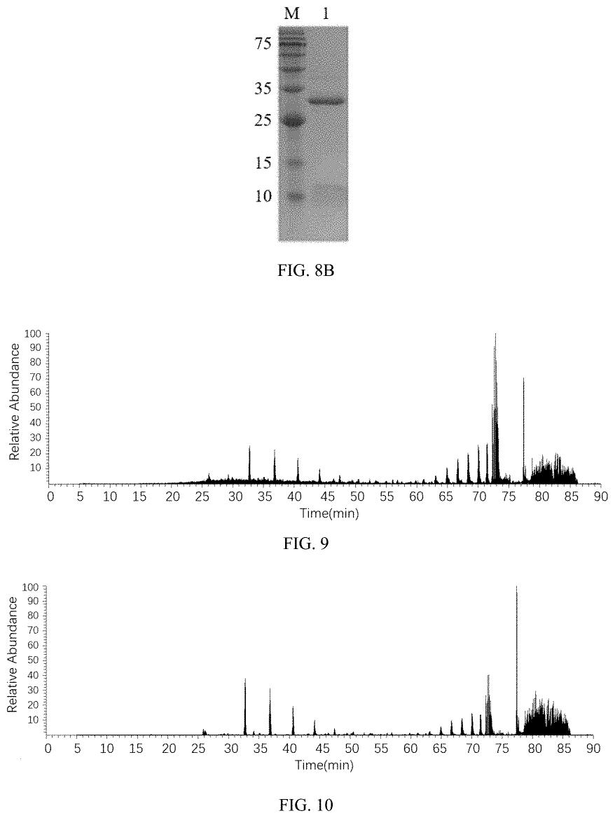

The purified inclusion bodies are subjected to in vitro renaturation with the random nonapeptide library. After molecular sieve chromatography (FIGS. 7A-7B) and ion exchange chromatography (FIGS. 8A-8B), SDS-PAGE revealed that the MHC class I-peptide complex is purified and highly concentrated, and could be used for binding peptide elution.

3.6 LC-MS/MS and De Novo Analysis

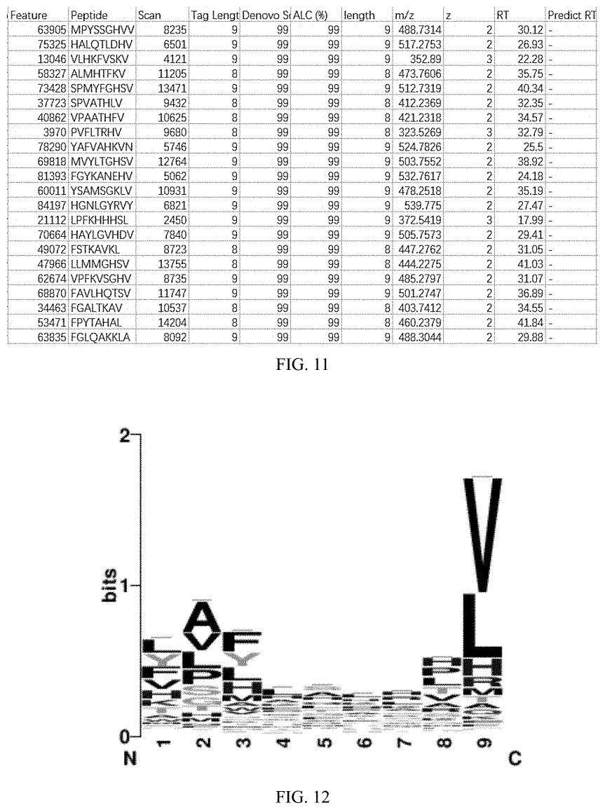

This analysis is mainly conducted by the Laboratory for Biological Mass Spectrometry, China Agricultural University. FIG. 9 shows the total ion chromatogram of the eluted peptide generated during mass spectrometry sequencing, FIG. 10 shows the Base Peak chromatogram of the eluted peptide generated during mass spectrometry sequencing, and FIG. 11 shows the results of the de novo analysis of the eluted peptide moiety.

3.7 Identification of the B2-Haplotype Chicken MHC I Binding Peptides Motif

The coefficient of variation (CV) of each binding site is calculated (the first to ninth positions at the N-terminal are 0.96, 1.21, 1.07, 0.65, 0.64, 0.56, 0.60, 0.78, and 2.11, respectively), and the second and ninth positions at the N-terminal are determined to be the restrictive sites. After the visual motif is drawn using the weblogo online tool, direct observation is performed. As shown in FIG. 12, two or three amino acids with the highest binding probability are selected as preferred amino acids, and the motif for selecting the candidate peptide epitopes is finally determined to be X-A/V-X-X-X-X-X-X-V/I/L. The qualified peptides in each protein sequence of ALV-J are screened according to this motif as the next test subjects (see Table 7).

3.8 Detection of Immunogenicity of Candidate Peptides by ELISPOT Assay

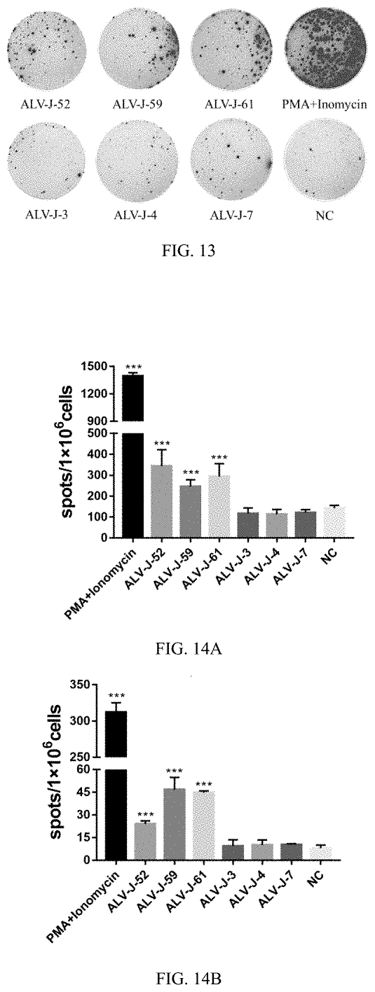

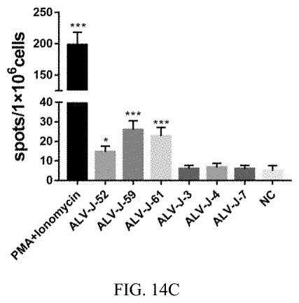

On the premise that the positive control group is established, ALV-J-52, ALV-J-59, ALV-J-61, ALV-J-3, ALV-J-4, and ALV-J-7 in the test group with the peptides as stimulants in Table 7 could generate spots compared with the negative control group. However, the experimental groups with ALV-J-52, ALV-J-59 and ALV-J-61 as stimulants significantly produce more spots (P<0.05), as shown in FIG. 13, indicating that these three peptides could stimulate cell secretion of IFN-γ, and are immunogenic T cell epitopes. The results of three independent replications are shown in FIGS. 14A-14C. The sequences of three qualified peptides identified are shown in SEQ ID NO:1 through SEQ ID NO:3 as shown in Table 8:

TABLE 8
Three Pieces of Information of ALV-J T Cell
Epitopes Against B2-haplotype Chicken

| | | Protein in | |
|---|---|---|---|
| | | which peptide | Protein |
| Peptide | Peptide sequence | is located | site |
| ALV-J-52 | TVDTASSAI | pol | 652-660 |
| | (SEQ ID NO: 1) | | |
| ALV-J-59 | FVDFANRLI | Gag | 403-411 |
| | (SEQ ID NO: 2) | | |
| ALV-J-61 | SALQAFREV | Gag | 374-382 |
| | (SEQ ID NO: 3) | | |

The above-mentioned embodiments are only for describing the preferred embodiments of the disclosure and are not intended to limit the scope of the disclosure. On the premise of not departing from the design spirit of the disclosure, various modifications and improvements made to the technical scheme of the disclosure by those of ordinary skill in the art should fall within the protection scope determined by the claims of the disclosure.

What is claimed is:

1. A J-subtype avian leukosis virus (ALV-J) B2-haplotype major histocompatibility complex (MHC-B2) restrictive epitope peptide, wherein an amino acid sequence of the ALV-J MHC-B2 restrictive epitope peptide is one selected from a group consisting of TVDTASSAI (SEQ ID NO:1), FVDFANRLI (SEQ ID NO:2) and SALQAFREV (SEQ ID NO:3).

2. An application method of the ALV-J MHC-B2 restrictive epitope peptide according to claim 1, wherein the ALV-J MHC-B2 restrictive epitope peptide is applied to prepare an ALV epitope-based vaccine.

3. A method for identifying the ALV-J MHC-B2 restrictive epitope peptide according to claim 1, comprising:
(1) obtaining an MHC class I molecular heavy chain protein and an MHC class I molecular light chain protein by using a prokaryotic expression system, mixing the MHC class I molecular heavy chain protein, the MHC class I light chain protein with a random peptide library and reacting to obtain an MHC class I-peptide complex;
(2) eluting the MHC class I-peptide complex obtained in the step (1) to obtain a peptide, sequencing and analyzing the peptide by a mass spectrum to determine a peptide restrictive epitope motif, and screening an ALV-J virus protein sequence according to the peptide restrictive epitope motif to obtain candidate peptides; and
(3) detecting immunogenicity of the candidate peptides in the step (2) by using a B2-haplotype animal model material infected with ALV-J virus, wherein the candidate peptides with the immunogenicity are the ALV-J MHC-B2 restrictive epitope peptides.

4. The method according to claim 3, wherein a molar ratio of the MHC class I molecular heavy chain protein:the MHC class I light chain protein:the random peptide library in step (1) is in a range of 1:1:(3-7).

5. The method of claim 3, wherein the random peptide library of the step (1) comprises one selected from a group consisting of an octapeptide library, a nonapeptide library, and a decapeptide library.

6. The method according to claim 3, wherein the mixing and reacting the MHC class I molecular heavy chain protein, the MHC class I light chain protein, and a random peptide library in step (1) specifically comprises:
adding the random peptide library into the MHC class I molecular light chain protein for reaction for 30 minutes after renaturation of the MHC class I molecular light chain protein, and then adding the MHC class I molecular heavy chain protein for renaturation.

7. The method according to claim 3, wherein a second position of an N-terminal of the peptide restrictive epitope motif in the step (2) is one of alanine (A or Ala) and valine (V or Val), and a ninth position of the N-terminal of the peptide restrictive epitope motif in the step (2) is one of valine (V or Val), isoleucine (I or Ile) and Leucine (L or Leu).

8. The method according to claim 3, wherein the detecting immunogenicity of the candidate peptides in the step (2) by using a B2-haplotype animal model material infected with ALV-J virus in the step (3) comprises:
performing an enzyme-linked immune absorbent spot (ELISpot) assay to screen the candidate peptides with immunogenicity by using the B2-haplotype animal model material infected with the ALV-J virus.


SEQUENCE LISTING (96 sequences)

SEQ ID NO: 1 - protein, 9 aa, Artificial Sequence
  ALV-J-52
TVDTASSAI

SEQ ID NO: 2 - protein, 9 aa, Artificial Sequence
  ALV-J-59
FVDFANRLI

SEQ ID NO: 3 - protein, 9 aa, Artificial Sequence
  ALV-J-61
SALQAFREV

SEQ ID NO: 4 - protein, 8 aa, Artificial Sequence
  gag
PALDWARI

SEQ ID NO: 5 - protein, 9 aa, Artificial Sequence
  gag
TAPLTDQGI

SEQ ID NO: 6 - protein, 9 aa, Artificial Sequence
  gag
IAAAMSSAI

SEQ ID NO: 7 - protein, 9 aa, Artificial Sequence
  gag
TANPQIHGI

SEQ ID NO: 8 - protein, 9 aa, Artificial Sequence
  gag
PVVAMPVVI

SEQ ID NO: 9 - protein, 9 aa, Artificial Sequence
  gag
GVINRDGSL

SEQ ID NO: 10 - protein, 9 aa, Artificial Sequence
  gag
EALMSFPLL

SEQ ID NO: 11 - protein, 9 aa, Artificial Sequence
  gag
AALLRPGEL

SEQ ID NO: 12 - protein, 9 aa, Artificial Sequence
  gag
QAFREVARL

SEQ ID NO: 13 - protein, 9 aa, Artificial Sequence
  gag
VAMVRGSIL

SEQ ID NO: 14 - protein, 9 aa, Artificial Sequence
  gag
MVLGKSGEL

SEQ ID NO: 15 - protein, 9 aa, Artificial Sequence
  gag
PVKQRSVYI

SEQ ID NO: 16 - protein, 9 aa, Artificial Sequence
  gag
VVDTANPQI

SEQ ID NO: 17 - protein, 9 aa, Artificial Sequence
  gag
AVAMVRGSI

SEQ ID NO: 18 - protein, 9 aa, Artificial Sequence
  gag
AALSQRAMV

SEQ ID NO: 19 - protein, 9 aa, Artificial Sequence
  gag
LVAIAASAL

SEQ ID NO: 20 - protein, 9 aa, Artificial Sequence
  gag
LASTGPPVV

SEQ ID NO: 21 - protein, 9 aa, Artificial Sequence
  gag
FANRLIKAV

SEQ ID NO: 22 - protein, 9 aa, Artificial Sequence
  gag
DVTNLMRVI

SEQ ID NO: 23 - protein, 9 aa, Artificial Sequence
  pol
VARCEQGAI

SEQ ID NO: 24 - protein, 9 aa, Artificial Sequence
  pol
RALSKACNI

SEQ ID NO: 25 - protein, 9 aa, Artificial Sequence
  pol
EASPLFAGI

SEQ ID NO: 26 - protein, 9 aa, Artificial Sequence
  pol
AVLGRPKAI

SEQ ID NO: 27 - protein, 9 aa, Artificial Sequence
  pol
GAVQQGAPV

SEQ ID NO: 28 - protein, 9 aa, Artificial Sequence
  pol
PARRFQWKV

SEQ ID NO: 29 - protein, 9 aa, Artificial Sequence
  pol
PALPLEGAV

SEQ ID NO: 30 - protein, 9 aa, Artificial Sequence
  pol
KAFTAWLEV

SEQ ID NO: 31 - protein, 9 aa, Artificial Sequence
  pol
SAFVAKMLL

SEQ ID NO: 32 - protein, 9 aa, Artificial Sequence
  pol
SAPALEAGV

SEQ ID NO: 33 - protein, 9 aa, Artificial Sequence
  pol
MAPRSWLAV

SEQ ID NO: 34 - protein, 9 aa, Artificial Sequence
  pol
AVRTFGKEV

SEQ ID NO: 35 - protein, 9 aa, Artificial Sequence
  pol
KVRVTDHPV

SEQ ID NO: 36 - protein, 9 aa, Artificial Sequence
  pol
NVVTDSAFV

SEQ ID NO: 37 - protein, 9 aa, Artificial Sequence
  pol
IVVTQHGRV

SEQ ID NO: 38 - protein, 9 aa, Artificial Sequence
  pol
TVLTEGPPV

SEQ ID NO: 39 - protein, 9 aa, Artificial Sequence
  pol
AALHLAIPL

SEQ ID NO: 40 - protein, 9 aa, Artificial Sequence
  pol
KASGSYRLL

SEQ ID NO: 41 - protein, 9 aa, Artificial Sequence
  pol
AAFILEDAL

SEQ ID NO: 42 - protein, 9 aa, Artificial Sequence
  pol
SAMAAVLHV

SEQ ID NO: 43 - protein, 9 aa, Artificial Sequence
  pol
SALPRGWPL

SEQ ID NO: 44 - protein, 9 aa, Artificial Sequence
  pol
LAASSHDGL

SEQ ID NO: 45 - protein, 9 aa, Artificial Sequence
  pol
VAEPRIATL

SEQ ID NO: 46 - protein, 9 aa, Artificial Sequence
  pol
PALGIPPRL

SEQ ID NO: 47 - protein, 9 aa, Artificial Sequence
  pol
MAWREIVQL

SEQ ID NO: 48 - protein, 9 aa, Artificial Sequence
  pol
TAWLEVLTL

SEQ ID NO: 49 - protein, 9 aa, Artificial Sequence
  pol
IARPLHVSL

SEQ ID NO: 50 - protein, 9 aa, Artificial Sequence
  pol
EARAVAMAL

SEQ ID NO: 51 - protein, 9 aa, Artificial Sequence
  pol
RAVAMALLL

SEQ ID NO: 52 - protein, 9 aa, Artificial Sequence
  pol
PVWIDQWPL

SEQ ID NO: 53 - protein, 9 aa, Artificial Sequence
  pol
AVQQGAPVL

SEQ ID NO: 54 - protein, 9 aa, Artificial Sequence
  pol
VVGQVLEPL

SEQ ID NO: 55 - protein, 9 aa, Artificial Sequence
  pol
QATFQAYPL

SEQ ID NO: 56 - protein, 9 aa, Artificial Sequence
  pol
EAKDLHTAL

SEQ ID NO: 57 - protein, 9 aa, Artificial Sequence
  pol
EAGVNPRGL

SEQ ID NO: 58 - protein, 8 aa, Artificial Sequence
  pol
MVERNRLL

SEQ ID NO: 59 - protein, 9 aa, Artificial Sequence
  pol
GVQYLGYKL

SEQ ID NO: 60 - protein, 9 aa, Artificial Sequence
  pol
DVQKLVGSL

SEQ ID NO: 61 - protein, 9 aa, Artificial Sequence
  GP85
LASQTACLI

SEQ ID NO: 62 - protein, 9 aa, Artificial Sequence
  GP85
TAKALPPGI

SEQ ID NO: 63 - protein, 9 aa, Artificial Sequence
  GP85
KALPPGIFL

SEQ ID NO: 64 - protein, 9 aa, Artificial Sequence
  GP85
NALGGPCYL

SEQ ID NO: 65 - protein, 9 aa, Artificial Sequence
  GP85
YVNRSWTMV

SEQ ID NO: 66 - protein, 9 aa, Artificial Sequence
  GP85
TACQNDTDL

SEQ ID NO: 67 - protein, 9 aa, Artificial Sequence
  GP37
AAAQALREI

SEQ ID NO: 68 - protein, 9 aa, Artificial Sequence
  GP37
AVLQNRAAI

SEQ ID NO: 69 - protein, 9 aa, Artificial Sequence
  GP37
RAAIDFLLL

SEQ ID NO: 70 - protein, 9 aa, Artificial Sequence
  GP37
LVIVCLLAI

SEQ ID NO: 71 - protein, 9 aa, Artificial Sequence
  GP37
FASFFAPGV

SEQ ID NO: 72 - protein, 9 aa, Artificial Sequence
  GP37
FALLVIVCL

SEQ ID NO: 73 - protein, 9 aa, Artificial Sequence
  GP37
QALREIERL

SEQ ID NO: 74 - protein, 9 aa, Artificial Sequence
  GP37
QANLTSLIL

SEQ ID NO: 75 - protein, 9 aa, Artificial Sequence
  GP37
LAKGVKTLL

SEQ ID NO: 76 - protein, 9 aa, Artificial Sequence
  GP37
GVKTLLFAL

SEQ ID NO: 77 - DNA, 20 bp, Artificial Sequence
  GAPDH
gaacatcatcccagcgtcca

SEQ ID NO: 78 - DNA, 20 bp, Artificial Sequence
  GAPDH
cggcaggtcaggtcaacaac

SEQ ID NO: 79 - DNA, 20 bp, Artificial Sequence
  Granzyme K
cgggaagcaactgttgaaat

SEQ ID NO: 80 - DNA, 20 bp, Artificial Sequence
  Granzyme K
gagtctcccttgcaagcatc

SEQ ID NO: 81 - DNA, 19 bp, Artificial Sequence
  Perforin
atggcgcaggtgacagtga

SEQ ID NO: 82 - DNA, 19 bp, Artificial Sequence
  Perforin
tggcctgcaccggtaattc

SEQ ID NO: 83 - DNA, 22 bp, Artificial Sequence
  IFN-gamma
cctccaacacctcttcaacatg

SEQ ID NO: 84 - DNA, 15 bp, Artificial Sequence
  IFN-gamma
tggcgtgcggtcaat

SEQ ID NO: 85 - DNA, 22 bp, Artificial Sequence
  TNF-alpha
gctgttctatgaccgcccagtt

SEQ ID NO: 86 - DNA, 21 bp, Artificial Sequence
  TNF-alpha
aacaaccagctatgcacccca

SEQ ID NO: 87 - DNA, 20 bp, Artificial Sequence
  IL-1beta
ggtcaacatcgccacctaca

SEQ ID NO: 88 - DNA, 22 bp, Artificial Sequence
  IL-1beta
catacgagatggaaaccagcaa

SEQ ID NO: 89 - DNA, 25 bp, Artificial Sequence
  IL-2
gctaatgactacagcttatggagca

SEQ ID NO: 90 - DNA, 23 bp, Artificial Sequence
  IL-2
tgggtctcagttggtgtgtagag

SEQ ID NO: 91 - DNA, 23 bp, Artificial Sequence
  NK lysin
gatggttcagctgcgtgggatgc

SEQ ID NO: 92 - DNA, 20 bp, Artificial Sequence
  NK lysin
ctgccggagcttcttcaaca

SEQ ID NO: 93 - DNA, 19 bp, Artificial Sequence
  HMG-2
agagcacaagaagaagcac

SEQ ID NO: 94 - DNA, 22 bp, Artificial Sequence
  HMG-2
gtcttttaggagcgttggggtc

SEQ ID NO: 95 - DNA, 20 bp, Artificial Sequence
  MHC-I
aagaaggggaagggctacaa

SEQ ID NO: 96 - DNA, 20 bp, Artificial Sequence
  MHC-I
aagcagtgcaggcaaagaat